(19) United States
(12) Patent Application Publication
(10) Pub. No.: US 2009/0053156 A1
(43) Pub. Date: Feb. 26, 2009

(54) HAIR SHAPING LOTION

(75) Inventor: Kazutoshi Kaizuka, Fukuoka (JP)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(73) Assignee: Kureitsu Co., Ltd., Fukuoka-shi (JP)

(21) Appl. No.: 11/817,483

(22) PCT Filed: Aug. 1, 2005
(86) PCT No.: PCT/JP2005/014482

§ 371 Date: Aug. 30, 2007

(30) Foreign Application Priority Data
Mar. 4, 2005 (JP) 2005-060537
May 20, 2005 (JP) 2005-148100

(51) Int. Cl. A61K 8/96

(52) U.S. Cl. 424/70.1

(57) ABSTRACT

A hair shaping lotion usable as a permanent wave agent or a hair straightener which contains a multi-element mineral selected from between perlite powder and fluorite powder and which can retain the shape of hair for a long period and is reduced in the damage to the hair and the irritation to the scalp or the fingers of an operator and excellent in hairstyle retention. Specifically, a hair shaping lotion which contains a multi-element mineral selected from between perlite powder and fluorite powder and 0.1 to 3.0 wt % of a reducing agent consisting of a mercapto compound, characterized by being made to act on the hair at 30 to 60° C. in the application. This lotion is used as the first lotion in combination with the second lotion containing hydrogen peroxide or a bromate.

### TECHNICAL FIELD

The present invention concerns a hairstyle formation and preservation preparation that is employed to form and preserve a hairstyle such as permanent wave hair or straight hair.

### BACKGROUND OF THE ART

Conventionally, it was necessary to use such hairstyle formation and preservation preparation as preparations for permanent waves or hair relaxer in order to obtain permanent wave hair or straight hair.

There have been the problems that these hairstyle formation and preservation preparations must compound a rather large amount of 4-12 wt % of a mercapto compound such as thioglycolic acid or cysteine as the chief preparation, and unpleasant odors, damage to the hair and irritation to the scalp occur, and the practitioners get rough, red hands.

To date, hair dyeing preparation compositions or a tooth brushing preparations that contain the pearlite powder, which is one of the multi-element minerals, have been disclosed for example in Unexamined Japanese Patent Application Number 2004-149464 and Unexamined Japanese Patent Application Number Hei 11-152216, but there is no trace of anything that contains pearlite powder or pitchstone powder, which are one kind of multi-element minerals, as the component of a hairstyle formation and preservation preparation.

The present inventions aims to provide a hairstyle formation and preservation preparation used for preparations for permanent waves and hair relaxers whereby the preservation of the hairstyle is good, there are no unpleasant odors, there is extremely little damage to the hair, the irritation to the scalp and to the practitioner's hands and fingers is low, and the preservation of the hairstyle is good, by including pearlite powder or pitchstone powder, which are one kind of multi-element minerals, in the components thereof.

### DISCLOSURE OF THE INVENTION

The inventive hairstyle formation and preservation preparation of the present invention contains pearlite powder or pitchstone powder, which are one kind of multi-element minerals, during use, and the hairstyle formation and preservation preparation of the present invention is taken as the first preparation, and this first preparation is used together with a second preparation that contains hydrogen peroxide and bromate. Table 1 shown below is one example of the composition table of pearlite, which is one of multi-element minerals.

The chemical composition of pitchstone is almost the same as pearlite except only for the fact that the crystal water contained in the raw stone differs. The hairstyle formation and preservation preparation of the present invention (first preparation) contains 0.001 to 10 wt %, preferably 0.01 to 0.2 wt %, and most preferably 0.03 to 0.1 wt %, pearlite powder or pitchstone powder, where in the pearlite or pitchstone is powderized. The hairstyle formation and preservation preparation of the present invention is used in a state in which the hair is heated at 30° C. to 60° C. with the far infrared promoters or the heating promoters like a hair steamer, or a hot curler, that is commonly used in beauty parlors. When the temperature of the hair is lower than 30° C., it is not possible to form permanent wave hair or straight hair, and when the temperature of the hair is higher than 60° C., this damages the hair.

TABLE 1

| | | |
|---|---|---|
| Silicic acid anhydride | (SiO₂) | 71.94% |
| Aluminum oxide | (Al₂O₃) | 14.94% |
| Iron oxide | (Fe₂O₃) | 2.54% |
| Magnesium oxide | (MgO) | 0.44% |
| Calcium oxide | (CaO) | 2.47% |
| Alkali oxide | (K₂O + Na₂O) | 6.87% |
| Manganese oxide | (MnO) | 0.03% |
| Phosphoric acid anhydride | (P₂O₅) | 0.14% |
| Loss on scorching | | 3.43% |
| Loss on drying (at 110° C.) | | 0.07% |
| Other titanium | | Traces |

The hairstyle formation and preservation preparation of the present invention (the first preparation) contains 0.1 to 3.0 wt %, preferably 1.5 to 2.5 wt %, and most preferably 2.0 wt %, of one kind or two or more kinds of mercapto compound as a reducing agent, for example, thioglycolic acid and its salts, L-cysteine and its salts, DL-cysteine and its salts, N-acetyl-L-cysteine, cysteamine and its salts, thiolactic acid and its salts, sulfite, bisulfite or thioglycine ester.

As the second preparation used in combination with the hairstyle formation and preservation preparation of the present invention (the first preparation), 0.01 to 10 wt % of hydrogen peroxide or bromate is included.

The hairstyle formation and preservation preparation of the present invention contains 0.01 to 10 wt % of one kind or two or more kinds of the alkali agents that are commonly used, for example, ammonia, monoethanol amine, diethanol amine, triethanol amine, aminomethyl propanol, carbonate or bicarbonate.

Other conventionally known components can be added to the hairstyle formation and preservation preparation of the present invention within a scope that does not hinder its results.

One can cite as other components the following: high class alcohols, aliphatic acids, silicone, peptides, amino acids, chelating agents, surfactants, sugars, pilatories, perfumes, etc.

### BEST MODE FOR EMBODYING THE INVENTION

A description is provided specifically of the embodiments of the present invention below, but the present invention is not limited to these embodiments.

### Embodiment 1

As shown in Table 2, the composition of the first preparation and the second preparation was adjusted for the four items, Invention Product 1, Invention Product 2, Invention Product 3 and the Comparative Product, and the wave formation rate based on the shrinkage rate was assessed for each item. Table 3 shows the results.

### (Assessment Method)

2 g of the first preparation was applied to approximately 2 g and 20 cm of a tuft of human hair, and after 15 minutes at 40° C. had passed, the second preparation was applied, and after it was left at room temperature for 5 to 10 minutes it was washed and dried.

TABLE 2

| | | Weight i concentration % | | | |
|---|---|---|---|---|---|
| | | Invention | Invention | Invention | Comparative |
| | | product 1 | product 2 | product 3 | example |
| First | Pearlite powder | 0.1 | 1 | 2 | — |
| preparation | Ammonium thioglycolate | 2 | 2 | 2 | 2 |
| | 25% ammonia water | 2 | 2 | 2 | 2 |
| | Cetostearyl alcohol | 2 | 2 | 2 | 2 |
| | Polyoxyethlyene cetyl | 0.5 | 0.5 | 0.5 | 0.5 |
| | ether | | | | |
| | Cetyltrimethylammonium | 1.0 | 1.0 | 1.0 | 1.0 |
| | chloride | | | | |
| | Purified water | To a total | To a total | To a total | To a total |
| | | amount of | amount of | amount of | amount of |
| | | 100 | 100 | 100 | 100 |
| Second | Sodium bromate | 4 | 4 | | 4 |
| preparation | Hydrogen peroxide | — | — | 1 | — |
| | pH adjusted water | To pH 7 | To pH 7 | To pH 3 | To pH 7 |
| | Purified water | To a total | To a total | To a total | To a total |
| | | amount of | amount of | amount of | amount of |
| | | 100 | 100 | 100 | 100 |

TABLE 3

| | Invention | Invention | Invention | Comparative |
|---|---|---|---|---|
| | product 1 | product 2 | product 3 | example |
| Wave effect | 87% | 80% | 76% | 96% |
| (shrinkage rate) | | | | |

The smaller the value for the shrinkage rate is, the higher the wave formation rate is.

As is clear from Table 3 above, Invention Product 1, Invention Product 2 and Invention Product 3, which contain pearlite powder, have a high wave formation rate, and possess superior elasticity and firmness.

Compared to this, the Comparative Product, which does not contain the pearlite powder, has a low wave formation rate compared with the Invention Products.

### Embodiment 2

As shown in Table 4, the composition of the first preparation and the second preparation was adjusted for the four items, Invention Product 4, Invention Product 5, Invention Product 6 and the Comparative Product, and the wave formation rate based on the shrinkage rate was assessed for each item. Table 5 shows the results.

### (Assessment Method)

2 g of the first preparation was applied to approximately 2 g and 20 cm of a tuft of human hair, and after 15 minutes at 40° C. had passed, the second preparation was applied, and after it was left at room temperature for 5 to 10 minutes it was washed and dried.

TABLE 4

| | | Weight i concentration % | | | |
|---|---|---|---|---|---|
| | | Invention | Invention | Invention | Comparative |
| | | product 4 | product 5 | product 6 | example |
| First | Pearlite powder | 0.1 | 1 | 2 | — |
| preparation | Ammonium thioglycolate | 2 | 2 | 2 | 2 |
| | 25% ammonia water | 2 | 2 | 2 | 2 |
| | Cetostearyl alcohol | 2 | 2 | 2 | 2 |
| | Polyoxyethlyne cetyl | 0.5 | 0.5 | 0.5 | 0.5 |
| | ether | | | | |
| | Cetyltrimethylammonium | 1.0 | 1.0 | 1.0 | 1.0 |
| | chloride | | | | |
| | Purified water | To a total | To a total | To a total | To a total |
| | | amount of | amount of | amount of | amount of |
| | | 100 | 100 | 100 | 100 |
| Second | Sodium bromate | 4 | 4 | | 4 |
| preparation | Hydrogen peroxide | — | — | 1 | — |
| | pH adjuster | To pH 7 | To pH 7 | To pH 3 | To pH 7 |
| | Purified water | To a total | To a total | To a total | To a total |
| | | amount of | amount of | amount of | amount of |
| | | 100 | 100 | 100 | 100 |

TABLE 5

| | Invention | Invention | Invention | Comparative |
|---|---|---|---|---|
| | product 4 | product 5 | product 6 | example |
| Wave effect | 85% | 81% | 78% | 96% |
| (shrinkage rate) | | | | |

The smaller the value for the shrinkage rate is, the higher the wave formation rate is.

As is clear from Table 5 above, Invention Product 4, Invention Product 5 and Invention Product 6, which contain pitchstone powder, have a high wave formation rate, and possess superior elasticity and firmness.

Compared to this, the Comparative Product, which does not contain the pitchstone powder, has a low wave formation rate compared with the Invention Products.

5 women between the ages of 20 and 40 were used as subjects A to D, and hairstyles were actually created with the hair of each subject by using the first preparations and second preparations adjusted to the composition shown in Table 2 and Table 4. Table 6 shows the results of the sensory assessment.

TABLE 6

| | | Wave or | | | |
|---|---|---|---|---|---|
| | | straight style | | | |
| | | formation | Texture | Bounce | Luster |
| A | Invention | ◯ | ◯ | ◯ | ◯ |
| | product 1 | | | | |
| | Invention | ◯ | ◯ | ◯ | ◯ |
| | product 2 | | | | |
| | Invention | ◯ | ◯ | ◯ | ◯ |
| | product 3 | | | | |
| | Comparative | X | Δ | X | Δ |
| | example | | | | |
| B | Invention | ◯ | ◯ | ◯ | ◯ |
| | product 4 | | | | |
| | Invention | ◯ | ◯ | ◯ | ◯ |
| | product 5 | | | | |
| | Invention | ◯ | ◯ | ◯ | ◯ |
| | product 6 | | | | |
| | Comparative | Δ | Δ | Δ | Δ |
| | example | | | | |
| C | Invention | ◯ | ◯ | ◯ | ◯ |
| | product 1 | | | | |
| | Invention | ◯ | ◯ | ◯ | ◯ |
| | product 2 | | | | |
| | Invention | ◯ | ◯ | ◯ | ◯ |
| | product 3 | | | | |
| | Comparative | X | Δ | X | Δ |
| | example | | | | |
| D | Invention | ◯ | ◯ | ◯ | Δ |
| | product 4 | | | | |
| | Invention | ◯ | ◯ | ◯ | ◯ |
| | product 5 | | | | |
| | Invention | ◯ | ◯ | ◯ | ◯ |
| | product 6 | | | | |
| | Comparative | Δ | X | X | X |
| | example | | | | |

As is clear from Table 6 above, the hairstyle formation and preservation preparation of the present invention has advantages over the Comparative Product in wave formation strength, touch, bounce and luster, and can create wave or straight hair styles that have bounce and luster without damaging the hair.

### POSSIBILITIES OF INDUSTRIAL APPLICATION

Since the hairstyle formation and preservation preparation of the present invention contains pearlite powder or pitchstone powder, which are one of the multi-element minerals, it is possible to reduce the amount of the mercapto compound that is used, and by taking this hairstyle formation and preservation preparation as the first preparation and using it in combination with a second preparation that contains hydrogen peroxide or bromate, it is possible to provide a hairstyle formation and preservation preparation with which the preservation of the hairstyle is good, the damage to the hair is extremely minor, the irritation to the scalp and the hands and fingers of the practitioner is low, and moreover the preservation of the hairstyle is good.

1. A hairstyle formation and preservation preparation that contains pearlite powder or pitchstone powder, which are one of the multi-element minerals.

2. The hairstyle formation and preservation preparation described in claim 1, which contains 0.1 to 3.0 wt % of a mercapto compound, which is the reducing agent.

3. The hairstyle formation and preservation preparation described in claim 1, which contains 1.5 to 2.5 wt % of a mercapto compound, which is the reducing agent.

4. The hairstyle formation and preservation preparation described in claim 1, which contains 2.0 wt % of a mercapto compound, which is the reducing agent.

5. The hairstyle formation and preservation preparation described in claim 1, characterized in that it is used by heating it to 30° C. to 60° C.
